(19) United States
(12) Patent Application Publication
(10) Pub. No.: US 2016/0081885 A1
(43) Pub. Date: Mar. 24, 2016

(54) POLYMERIZABLE DENTAL MATERIAL

(71) Applicant: Muhlbauer Technology GmbH, Hamburg (DE)

(72) Inventors: Stephan Neffgen, Pinneberg (DE); Silke Ziegler, Hamburg (DE)

(21) Appl. No.: 14/863,879

(22) Filed: Sep. 24, 2015

(30) Foreign Application Priority Data
Sep. 24, 2014 (DE) 102014219218.8

(51) Int. Cl. A61K 6/00

(52) U.S. Cl. CPC A61K 6/0023

(57) ABSTRACT

A subject matter of the invention is a self-etching self-curing dental adhesive which, through the use of a specific redox initiator system comprising at least six constituents, on the one hand is stable on storage as two-component kit and, on the other hand, shows a high shear bond strength on use.

The invention relates to a polymerizable dental material, in particular a self-etching self-curing dental adhesive, to a kit for the preparation of such a dental material and to a complex redox initiator system for a two-part self-etching self-curing dental adhesive with improved adhesion.

US2013/0143176 describes a kit of at least two parts for the preparation of a dental adhesive. A first part comprises at least monomer comprising acid groups and initiator. The initiator typically consists at least of one of the elements oxidizing agent, reducing agent, sensitizer, onium salt, electron donor or of a combination of two or more of these elements. Photoinitiators are preferred sensitizers. A second part comprises an activator for these initiators which is incorporated in a polymer film and is released from this in delayed fashion on contact with the first part. Sodium toluenesulfinate and sodium benzenesulfinate are particularly preferred activators. When a brush exhibiting such a film is employed five times in succession, the shear bond strength of a resin cement on etched dentin is improved.

US2010/0129777 describes a three-part kit. A first part comprises at least one monomer comprising acid groups and a preferably organic peroxide as initiator (oxidizing agent). A second part is acid-free and comprises monomers and also a photoinitiator, a first tertiary aromatic amine without a benzoic acid ester group (reducing agent for the oxidizing agent) and also a second tertiary aromatic amine with a benzoic acid ester group (reducing agent for the photoinitiator). A third part comprises ethanol and sodium benzenesulfinate. The kit is to be used in such a way that first the electron donor is applied to etched dentin, followed by a mixture of the two other parts. This method led to good shear bond strength with a resin composite.

US2010/0087613 describes a two-part adhesive material. A first part comprises at least one monomer comprising acid groups and a peroxide. A second part is acid-free and comprises monomers and also aromatic sulfinate in the powder form, a photoinitiator and a tertiary aromatic amine.

In order to be able to store the redox initiator systems of a self-etching dental adhesive for as long as possible and to achieve the desired adhesion, these are stored and used in three parts. It is also known to enclose photoinitiators with two-part dental materials comprising redox initiator systems, such as peroxide/amine or hydroperoxide/thiourea, whereby they, where it is possible, can also be polymerized by means of light activation, which generally improves the adhesion. However, there further exists a need for self-etching self-curing dental adhesives which are stable on storage, simple and safe to use.

It is an object of the invention to create a dental material, a kit and a redox system of the type mentioned at the start which are stable on storage and also can be applied simply and safely.

A dental material according to the invention comprises the following constituents:
a. at least one monomer, oligomer or prepolymer comprising at least one radically polymerizable group and at least one acid group,
b. at least one monomer, oligomer or prepolymer which does not comprise any acid groups and comprises at least one radically polymerizable group,
c. at least one solvent,
d. at least three oxidizing agents comprising a peroxo group chosen from the group consisting of organic peroxides, organic hydroperoxides and persulfates,
e. at least one sulfinic acid and/or one sulfinic acid salt,
f. at least one photoinitiator,
g. at least two tertiary amines, at least one of which exhibits a methyl benzoate group or ethyl benzoate group and at least one of which does not exhibit a methyl benzoate group or an ethyl benzoate group.

The invention has recognized that, surprisingly, the specific redox initiator system claimed, as defined in the characteristics d) to g), results in a dental material, in particular dental adhesive, which (optionally in components of a kit) before use is storable in the nonpolymerized state, achieves very good coefficients of adhesion, in particular on dentin, and is developed in a self-curing or binary hardening fashion. The invention has recognized that, surprisingly, even the photoinitiator contributes to an improved adhesion. This is even valid if the dental material is used in self-curing fashion, i.e. is cured without the action of light, as is shown in the comparative examples. This discovery is especially surprising. The redox initiator system comprises oxidizing agent comprising at least three different peroxo compounds, reducing agent comprising sulfinic acid and/or sulfinic acid salt, a first tertiary amine and a second tertiary amine comprising a methyl benzoate or ethyl benzoate group.

First, some terms used in the context of the invention are explained. Self-etching means that the bonding surface of the dentin does not need to be etched in a separate stage by means of acid in order to achieve good coefficients of adhesion. Self-curing means that the polymerization is activated purely chemically by mixing the components. The invention makes available such a self-etching self-curing dental adhesive. The patent claims directed toward the dental material claim the material in the not yet polymerized or cured state.

Preferred constituents a) are radically polymerizable monomers which exhibit at least one acid group and at least one vinyl, (meth)acrylamide or, preferably, (meth)acrylate group. Preferred acid groups are carboxylic acid groups, carboxylic acid anhydride groups, phosphonic acid groups or, particularly preferably, phosphoric acid groups.

Suitable radically polymerizable monomers which exhibit at least one carboxylic acid group or carboxylic acid anhydride group are, for example, vinylbenzoic acid, (meth)acrylic acid, (meth)acryloyloxydecylmalonic acid, maleic acid monohemiester, N-(2-hydroxy-3-(meth)acryloyloxypropyl)-N-phenylglycine, 4-((meth)acryloylamino)salicylic acid, maleic acid, itaconic acid, maleic anhydride, 4-((meth)acryloyloxyethyl)trimellitic acid, 4-((meth)acryloyloxyethyl)trimellitic anhydride or N-((meth)acryloyl-N′,N′-dicarboxymethyl-1,4 diaminobenzene.

Further suitable radically polymerizable monomers which exhibit at least one carboxylic acid group are addition products of a carboxylic acid anhydride, for example maleic anhydride, phthalic anhydride, pyromellitic anhydride or 3,3′,4,4′-benzophenonetetracarboxylic dianhydride, and of a hydroxyalkyl(meth)acrylate, for example 2-hydroxyethyl(meth)acrylate and glycerol di(meth)acrylate.

Suitable radically polymerizable oligomers or prepolymers which exhibit at least one carboxylic acid group or carboxylic acid anhydride group are homopolymers or copolymers of unsaturated carboxylic acids, for example (meth)acrylated polycarboxylic acids of the carboxylic acids (meth)acrylic acid, maleic acid, maleic anhydride and/or itaconic acid. Suitable radically polymerizable homopolymers or copolymers preferably exhibit an average molar mass of 2000 to 100 000, preferably 5 000 to 60 000, more preferably 10 000 to 50 000, and more preferably 20 000 to 40 000.

Suitable radically polymerizable monomers which exhibit at least one phosphoric or phosphonic acid group are vinylbenzylphosphonic acid, methacryloyloxyethyl dihydrogenphosphate, methacryloyloxypropyl dihydrogenphosphate, methacryloyloxyhexyl dihydrogenphosphate, glyceryl dimethacrylate phosphate, pentaerythritol triacrylate phosphate, bis(hydroxyethyl methacrylate) phosphate, phenyl methacryloyloxyalkyl phosphates, (meth)acrylamidophosphates, (meth)acrylamidodiphosphates, bismethacrylamidoalkyl dihydrogenphosphates or (meth)acrylamidoalkyl diphosphonates. Methacryloyloxydecyl dihydrogenphosphate (MDP) is particularly preferred.

Suitable constituents b) are radically polymerizable monomers, oligomers or prepolymers which exhibit no acid group and at least one (meth)acrylate, (meth)acrylamide or vinyl group. Monomers, in particular monomers with (meth)acrylate groups, are preferred. Mention may be made, by way of example, of methyl(meth)acrylate, ethyl(meth)acrylate, propyl(meth)acrylate, butyl(meth)acrylate, hexyl(meth)acrylate, octyl(meth)acrylate, lauryl(meth)acrylate, decyl(meth)acrylate, tridecyl(meth)acrylate, 2-ethoxyethyl(meth)acrylate, 2′-ethoxy-2-ethoxyethyl(meth) acrylate, ethylene glycol di(meth)acrylate, polyethylene glycol mono(meth)acrylate, polypropylene glycol mono(meth)acrylate, polytetramethylene glycol mono(meth)acrylate, diethylene glycol di(meth)acrylate, triethylene glycol di(meth)acrylate, tetraethylene glycol di(meth)acrylate, polyethylene glycol di(meth)acrylate, polypropylene glycol di(meth)acrylate, polytetramethylene glycol di(meth)acrylate, hexanediol di(meth)acrylate, UDMA (reaction product of 2-hydroxyethyl methacrylate with 2,4,4-trimethylhexane diisocyanate), 2,2-bis[4-(2-hydroxy-3-methacryloyloxypropoxy)phenyl]propane (Bis-GMA), ethoxylated bisphenol A di(meth)acrylate and the like.

Preferred radically polymerizable monomers which exhibit no acid group comprise at least two (meth)acrylate, (meth)acrylamide or vinyl groups (crosslinking agents).

Preferred radically polymerizable monomers which exhibit no acid group form, on the tooth substance, a film which essentially covers the surface without any gaps (film-forming agent). A film-forming agent is preferably at least so readily soluble in water that a solution of 5% by weight of this film-forming agent in water can be prepared. It is preferably completely miscible with water. Film-forming agents preferably comprise hydrophilic groups, in particular hydroxyl groups. The film-forming agent preferably comprises copolymerizable groups, preferably vinyl groups, (meth)acrylamide groups and (meth)acrylate groups.

Preferred film-forming agents are 2-hydroxyethyl acrylate, 2-hydroxyethyl methacrylate (HEMA), 2- and 3-hydroxypropyl acrylate and methacrylate, 1,3- and 2,3-dihydroxypropyl acrylate and methacrylate, 2-hydroxypropyl-1,3-diacrylate and -dimethacrylate, 3-hydroxypropyl-1,2-diacrylate and -dimethylacrylate, hydroxybutyl(meth)acrylate, pentaerythritol diacrylate and dimethacrylate, 2-trimethylammonioethyl methacrylate chloride, 2-hydroxyethylacrylamide and -methacrylamide, N,N-bis(2-hydroxyethyl)acrylamide and -methacrylamide, N-alkyl-N-(hydroxyethyl)acrylamide and -methacrylamide, 2- and 3-hydroxypropylacrylamide and -methacrylamide, methacrylamidopropyltrimethylammonium chloride, O-methacryloyltyrosinamide and 2-acrylamido-2-methylpropanesulfonic acid.

Particularly preferred film-forming agents are 2-hydroxymethyl methacrylate; 2- and 3-hydroxypropyl acrylate; 2- and 3-hydroxypropyl methacrylate; 1,3- and 2,3-dihydroxypropyl acrylate; and 1,3- and 2,3-dihydroxypropyl methacrylate.

The self-etching self-curing dental adhesive comprises from 1 to 50% by weight of constituent a), preferably from 5 to 20% by weight.

The self-etching self-curing dental adhesive comprises from 5 to 95% by weight of constituent b), preferably from 5 to 90% by weight.

The self-etching self-curing dental adhesive comprises from 1 to 90% by weight of solvents, preferably from 10 to 80% by weight.

Suitable solvents (constituent c)) are preferably inert (radically nonpolymerizable) under the conditions of use and are preferably chosen from physiologically acceptable solvents. Suitable solvents are, for example, water, acetone or alkyl alcohols, such as ethanol, isopropanol or 2-methyl-2-propanol. It is preferable for the physiologically acceptable solvent to be readily volatile at body temperature, such as, for example, ethanol. “Physiologically acceptable” means, in the context of the invention, that the solvent in normal application has no or only insignificant disadvantageous effects on the organism.

Suitable constituents d) are the persulfate, organic peroxide and organic hydroperoxide oxidizing agents.

Suitable persulfates are, for example, sodium persulfate, potassium persulfate and ammonium persulfate. The proportion of persulfate, based on the total weight of the polymerizable constituents a) and b), is preferably from 0.05 to 2% by weight.

Suitable organic peroxides are, for example, dipropyl peroxide, dibutyl peroxide, dilauryl peroxide, diacetyl peroxide, dicapryl peroxide, dibenzoyl peroxide (BPO), p,p′-dichlorobenzoyl peroxide, p,p′-dimethoxybenzoyl peroxide, p,p′-dimethylbenzoyl peroxide, p,p′-dinitrodibenzoyl peroxide and di(3,5,5-trimethylhexanoyl)peroxide. Dibenzoyl peroxide (BPO) is preferred. The proportion of organic peroxide, based on the total weight of the polymerizable constituents a) and b), is preferably from 0.1 to 5% by weight.

Suitable organic hydroperoxides are, for example, 1,1-dimethylethyl hydroperoxide, 1,1-dimethylpropyl hydroperoxide, 1,1-dimethylbutyl hydroperoxide, cumene hydroperoxide, p-menthane hydroperoxide, pinene hydroperoxide, 1,1,3,3-tetramethylbutyl hydroperoxide, diisopropylbenzene monohydroperoxide, p-isopropylcumene hydroperoxide, p-(tert-butyl)cumene hydroperoxide, cyclohexyl hydroperoxide, methylcyclohexane hydroperoxide, tetralin hydroperoxide and 1,1-diphenylethane hydroperoxide. The proportion of organic hydroperoxide, based on the total weight of the polymerizable constituents a) and b), is preferably from 0.1 to 5% by weight.

Suitable constituents e) are organic sulfinic acids which function as electron donors, such as alkanesulfinic acids (for example ethanesulfinic acid, propanesulfinic acid, hexanesulfinic acid, octanesulfinic acid, decanesulfinic acid and dodecanesulfinic acid); alicyclic sulfinic acids (for example cyclohexanesulfinic acid and cyclooctanesulfinic acid); and also aromatic sulfinic acids (for example benzenesulfinic acid, o-toluenesulfinic acid, p-toluenesulfinic acid, ethylbenzenesulfinic acid, decylbenzenesulfinic acid, dodecylbenzenesulfinic acid, chlorobenzenesulfinic acid and naphthalenesulfinic acid).

Additional suitable constituents e) are salts of the organic sulfinic acids, such as alkali metal salts, alkaline earth metal salts, amine salts or ammonium salts. The alkali metal salts can in particular be lithium, sodium or potassium salts. Among the alkaline earth metal salts, magnesium, calcium, strontium and barium salts are preferred. The amine salts can be primary amine salts which, for example, exhibit the following groups: methylamine, ethylamine, propylamine, butylamine, aniline, toluidine, phenylenediamine or xylylenediamine. Secondary amine salts can exhibit the following groups: dimethylamine, diethylamine, dipropylamine, dibutylamine, piperidine, N-methylaniline, N-ethylaniline, diphenylamine or N-methyltoluidine. Tertiary amines can exhibit the following groups: trimethylamine, triethylamine, pyridine, N,N-dimethylaniline, N,N-di(β-hydroxyethyl)aniline, N,N-diethylamine, N,N-dimethyltoluidine, N,N-diethyltoluidine or N,N-di(β-hydroxyethyl)toluidine. Tetramethylammonium salts, tetraethylammonium salts, tetrapropylammonium salts or trimethylbenzylammonium salts, for example, can be used as ammonium salts. Suitable organic sulfinic acid salts are, for example, benzenesulfinic acid salts with the following counterions: sodium, potassium, magnesium, calcium, strontium, barium, butylamine, aniline, toluidine, phenylenediamine, diethylamine, diphenylamine, triethylamine, ammonium, tetramethylammonium and trimethylbenzylammonium. Suitable counterions for o-toluenesulfinic acid salts are, for example, lithium, sodium, potassium, calcium, cyclohexylamine, aniline, ammonium and tetraethylammonium. Suitable counterions for p-toluenesulfinic acid salts are, for example, lithium, sodium, potassium, calcium, barium, ethylamine, toluidine, N-methylaniline, pyridine, ammonium and tetramethylammonium. Suitable counterions for p-naphthalenesulfinic acid salts are, for example, sodium, strontium, triethylamine, N-methyltoluidine, ammonium, trimethylbenzylammonium and the like.

Particularly preferred aromatic sulfinate salts are sodium benzenesulfinate and sodium toluenesulfinate.

The proportion of organic sulfinic acids or the salts thereof, based on the total weight of the polymerizable constituents a) and b), is preferably from 0.5 to 5% by weight.

Suitable photoinitiators (constituent f)) induced by visible light, preferably blue visible light, are known to a person skilled in the art. Mention may be made, by way of example, of camphorquinone, 1-phenylpropane-1,2-dione, benzil, diacetyl, benzil dimethyl ketal, benzil diethyl ketal, benzil di(2-methoxyethyl)ketal, anthraquinone, 1-chloroanthraquinone, 1-chloroanthraquinone, 1,2-benzanthraquinone, 1-hydroxyanthraquinone, 1-methylanthraquinone, 2-ethylanthraquinone, 1-bromoanthraquinone, thioxanthone, 2-isopropylthioxanthone, 2-nitrothioxanthone, 2-methylthioxanthone, 2,4-dimethylthioxanthone, 2,4-diethylthioxanthone, 2,4-diisopropylthioxanthone, 2-chloro-7-(trifluoromethyl)thioxanthone, thioxanthone 10,10-dioxide, thioxanthone 10-oxide, benzoin methyl ether, benzoin ethyl ether, benzoin isopropyl ether, benzoin isobutyl ether, benzophenone, bis(4-dimethylaminophenyl)ketone, 4,4′-bis(diethylamino)benzophenone, acylphosphine oxides, such as (2,4,6-trimethylbenzoyl)diphenylphosphine oxide or bis(2,4,6-trimethylbenzoyl)phenylphosphine oxide, benzoyltrimethylgermanium, dibenzoyldiethylgermanium and diaryliodonium salts, such as diphenyliodonium hexafluorophosphate, diphenyliodonium hexafluoroantimonate, bis(4-bromophenyl)iodonium triflate, bis(4-tert-butylphenyl)iodonium hexafluorophosphate, bis(4-fluorophenyl)iodonium triflate, bis(4-methylphenyl)iodonium hexafluorophosphate, (2-bromophenyl)(2,4,6-trimethylphenyl)iodonium triflate, (3-bromophenyl)(2,4,6-trimethylphenyl)iodonium triflate, (2-methylphenyl)(2,4,6-trimethylphenyl)iodonium triflate, (3-methylphenyl)(2,4,6-trimethylphenyl)iodonium triflate, (4-methylphenyl)(2,4,6-trimethylphenyl)iodonium triflate, (4-nitrophenyl)phenyliodonium triflate, (4-nitrophenyl)(2,4,6-trimethylphenyl)iodonium triflate, phenyl[3-(trifluoromethyl)phenyl]iodonium triflate and [3-(trifluoromethyl)phenyl](2,4,6-trimethylphenyl)iodonium triflate. Camphorquinone is preferred.

The proportion of photoinitiators, based on the total weight of the polymerizable constituents a) and b), is preferably from 0.1 to 5% by weight.

Suitable reducing agents (constituents g)) are combinations of at least two tertiary amines. Suitable first tertiary amines are aliphatic, alicyclic or aromatic tertiary amines, the tertiary aromatic amines exhibiting, on a benzene ring, in the para position with respect to the amine group, a group with a positive inductive effect. Suitable are, for example, N,N-bis(2-hydroxyethyl)-p-toluidine, 2-[4-(dimethylamino)phenyl]ethanol and N,N-dimethyl-p-(tert-butyl)aniline. The proportion of first tertiary amine, based on the total weight of the polymerizable constituents a) and b) and optionally present film-forming agents (dependent claim 4), is preferably from 0.05 to 2% by weight. Suitable second tertiary amines are aromatic tertiary amines comprising a methyl benzoate group or an ethyl benzoate group, preferably in the para position. Preferred compounds are methyl N,N-dimethylaminobenzoate and ethyl N,N-dimethylaminobenzoate. The proportion of second tertiary amine, based on the total weight of the polymerizable constituents a) and b), is preferably from 0.5 to 5% by weight.

The self-etching self-curing dental adhesive is preferably stored as a kit in two components A and B separated from one another and held available for application. Component A comprises at least the constituents a) and d) and component B comprises at least the constituents c), e) and g). Component A preferably comprises the constituents a), b) and d) and component B preferably comprises the constituents c), e), f) and g). Preferably, none of the radically polymerizable constituents a) and b) are present in component B of the kit. Both components can comprise additives, such as stabilizers, fillers, and the like, customary in dentistry.

An additional subject matter of the invention is the use of a redox initiator system which comprises:
d. at least three oxidizing agents comprising a peroxo group chosen from the group consisting of organic peroxides, organic hydroperoxides and persulfates,
e. at least one sulfinic acid and/or one sulfinic acid salt,
f. at least one photoinitiator,
g. at least two tertiary amines, at least one of which exhibits a methyl benzoate group or ethyl benzoate group and at least one of which does not exhibit a methyl benzoate group or an ethyl benzoate group;
as initiator system in a polymerizable dental material, preferably a self-etching dental adhesive.

A subject matter of the invention is likewise the use of a dental material according to the invention as self-etching dental adhesive or the use of a kit according to the invention for the preparation of such a self-etching dental adhesive. The abovementioned use or preparation is preferably carried out by the dentist at or in the vicinity of the site of the treatment.

### EXAMPLES

Subsequently, a dental material according to the invention is compared with 15 comparative examples, in which each time modifications not according to the invention were carried out, such as, for example, the exclusion or exchange of a constituent of the dental material according to the invention.

| | C12 | C11 | C10 | C13 | C14 | C15 | Example | C3 | C7 | C8 | C9 | C6 | C1 | C2 | C5 | C4 |
|---|---|---|---|---|---|---|---|---|---|---|---|---|---|---|---|---|
| SBS | 5.2 ± | 5.9 ± | 2.5 ± | 5.2 ± | 5 ± | 5.5 ± | 8.8 ± | 3.3 ± | 4.1 ± | 5.6 ± | 3.1 ± | 4.5 ± | 3.8 ± | 0 | 0 | 0 |
| | 2.5 | 1.7 | 1.4 | 2.5 | 3.0 | 2.0 | 2.3 | 2.3 | 2.1 | 2.5 | 0.7 | 1.6 | 1.8 | | | |

Component A [% by weight]

| | | | | | | | | | | | | | | | | |
|---|---|---|---|---|---|---|---|---|---|---|---|---|---|---|---|---|
| MDP | 16.5 | 16.3 | 16.3 | 16.1 | 16.1 | 15.8 | 15.7 | 15.7 | 15.7 | 15.7 | 15.7 | 15.7 | 15.7 | 15.7 | 15.7 | 15.7 |
| BisGMA | 37.6 | 37 | 37 | 36.6 | 36.6 | 36.1 | 35.7 | 35.7 | 35.7 | 35.7 | 35.7 | 35.7 | 35.7 | 35.7 | 35.7 | 35.7 |
| HEMA | 44.7 | 44.1 | 44.1 | 43.6 | 43.6 | 42.9 | 42.5 | 42.5 | 42.5 | 42.5 | 42.5 | 42.5 | 42.5 | 42.5 | 42.5 | 42.5 |
| BPO | — | 2.5 | — | 2.5 | — | 2.5 | 2.5 | 2.5 | 2.5 | 2.5 | 2.5 | 2.5 | 2.5 | 2.5 | 2.5 | 2.5 |
| Na₂S₂O₈ | 1 | — | — | 1 | 1 | | 1 | 1 | 1 | 1 | 1 | 1 | 1 | 1 | 1 | 1 |
| AHP | — | — | 2.5 | — | 2.5 | 2.5 | 2.5 | 2.5 | 2.5 | 2.5 | 2.5 | 2.5 | 2.5 | 2.5 | 2.5 | 2.5 |

Component B [% by weight]

| | | | | | | | | | | | | | | | | |
|---|---|---|---|---|---|---|---|---|---|---|---|---|---|---|---|---|
| EtOH/H₂O (2:1) | 92 | 92 | 92 | 92 | 92 | 92 | 92 | 92.6 | 93.6 | 96.4 | 97 | 94.8 | 95 | 95 | 95 | 99.4 |
| CQ | 1.6 | 1.6 | 1.6 | 1.6 | 1.6 | 1.6 | 1.6 | 1.6 | — | — | — | 1.6 | 1.6 | 1.6 | 1.6 | — |
| Na sulfinate | 3 | 3 | 3 | 3 | 3 | 3 | 3 | 3 | 3 | 3 | 3 | 3 | — | — | — | — |
| EDMAB | 2.8 | 2.8 | 2.8 | 2.8 | 2.8 | 2.8 | 2.8 | 2.8 | 2.8 | — | — | — | 2.8 | 2.8 | — | — |
| EHA | — | — | — | — | — | — | — | — | — | — | — | — | — | — | 2.8 | — |
| DHEPT | 0.6 | 0.6 | 0.6 | 0.6 | 0.6 | 0.6 | 0.6 | — | — | 0.6 | — | 0.6 | 0.6 | — | 0.6 | 0.6 |
| DMPTB | — | — | — | — | — | — | — | — | 0.6 | — | — | — | — | 0.6 | — | — |

For the preparation of components A and B, the constituents given in the table were stirred with a magnetic stirrer for a long enough period of time until a homogeneous mixture was formed.

The following constituents were used:

| | |
|---|---|
| BisGMA | Bisphenol A diglycidyl |
| | dimethacrylate (CAS 001565-94-2) |
| MDP | Methacryloyloxydecyl phosphate |
| HEMA | 2-Hydroxyethyl methacrylate |
| BHT | 2,6-Di(tert-butyl)-4-methylphenol |
| AHP | tert-Amyl hydroperoxide |
| BPO | Dibenzoyl peroxide |
| Na₂S₂O₈ | Sodium persulfate |
| EtOH | Ethanol |
| CQ | Camphorquinone |
| EHA | 2-Ethylhexyl |
| | p-dimethylamino)benzoate |
| EDMAB | Ethyl dimethylaminobenzoate |
| DHEPT | N,N-Bis(2-hydroxyethyl)-p-toluidine |
| DMPTB | N,N-Dimethyl-p-(tert-butyl)aniline |
| Na sulfinate | Sodium benzenesulfinate |

The tests were carried out according to ISO/TS 11405:2003 (Dental materials—Testing of adhesion to tooth structure), unless otherwise indicated.

First, bovine dentin surfaces were prepared. Then the adhesive was prepared by adding components A and B in the ratio of 1:1 (volumes) to a mixing palette and mixing for 5 s. The adhesive thus prepared was subsequently each time applied to and worked into the unetched bovine dentin surfaces for 20 s. Blowing with compressed air was then carried out for 10 s. This operation was repeated again. Immediately thereafter, a two-part Teflon mold (according to ISO/TS 11405:2003) was put on and a self-curing dental composite (Luxacore® Dual, DMG) was introduced. The dental composite was allowed to cure under laboratory conditions (23° C., yellow room) for 12 min. The Teflon mold was removed and the adhesivated tooth was stored in water at 37° C. for 24 h. Subsequently, the shear bond strength (SBS) was measured. Ten individual measurements per adhesive were carried out.

It can be inferred, from the following table, comprising the compositions of the adhesives and the results of the shear bond measurements, that the specific combination of at least three different oxidizing agents with at least one sulfinic acid salt, at least one photoinitiator and at least two different tertiary amines, at least one of the tertiary amines exhibiting a short-chain benzoic acid ester group, results in a significantly improved shear bond strength.

As is shown by comparative examples C1 to C15, this combination according to the invention is not in the least random or only concentration-dependent. In particular, it was surprising that the amine EDMAB, which is normally used for the activation of camphorquinone, had a positive effect, although no light curing was carried out.

1. A dental material, which comprises:
a) at least one monomer, oligomer or prepolymer comprising at least one radically polymerizable group and at least one acid group,
b) at least one monomer, oligomer or prepolymer which does not comprise any acid groups and comprises at least one radically polymerizable group,
c) at least one solvent,
d) at least three oxidizing agents comprising a peroxo group chosen from the group consisting of organic peroxides, organic hydroperoxides and persulfates,
e) at least one sulfinic acid and/or one sulfinic acid salt,
f) at least one photoinitiator,
g) at least two tertiary amines, at least one of which exhibits a methyl benzoate group or ethyl benzoate group and at least one of which does not exhibit a methyl benzoate group or an ethyl benzoate group.

2. The dental material as claimed in claim 1, wherein the acid group in the constituent a) is chosen from the group consisting of carboxylic acid, phosphonic acid, and phosphoric acid groups.

3. The dental material as claimed in claim 1, wherein constituent b) comprises a film-forming agent.

4. The dental material as claimed in claim 3, wherein the film-forming agent is soluble in water to at least 5% by weight.

5-14. (canceled)

15. The dental material as claimed in claim 4, wherein the film-forming agent is miscible with water.

16. The dental material as claimed in claim 1, wherein constituent b) comprises monomers, oligomers or prepolymers with at least two radically polymerizable groups.

17. The dental material as claimed in claim 16, wherein the radically polymerizable groups are chosen from the group consisting of acrylate, methacrylate, acrylamide, methacrylamide, and vinyl groups.

18. The dental material as claimed in claim 1, wherein the solvent c) is chosen from the group consisting of water, alcohols, and acetone.

19. The dental material as claimed in claim 1, which comprises, based on the total weight of polymerizable constituents, one or more of the constituents mentioned below in the constituent amounts mentioned below:
from 0.05 to 2% by weight of persulfates,
from 0.1 to 5% by weight of organic peroxides,
from 0.1 to 5% by weight of organic hydroperoxides,
from 0.5 to 5% by weight of sulfinic acid and/or sulfinic acid salt,
from 0.1 to 5% by weight of photoinitiator.

20. The dental material as claimed in claim 1, wherein the dental material is a self-etching self-curing dental adhesive.

21. A method of adhering to a dental surface comprising using the dental material as claimed in claim 1 as self-etching dental adhesive.

22. A kit for the preparation of a dental material as claimed in claim 1, the kit comprising at least two components A and B, component A comprising at least the constituents a) and d) and component B comprising at least the constituents c), e) and g).

23. The kit as claimed in claim 22, wherein component A comprises the constituents a), b), d) and also optionally present film-forming agents and wherein component B comprises the constituents c), e), f) and g).

24. A method of preparing a self-etching dental adhesive comprising using a kit as claimed in claim 22 in the preparation of a self-etching dental adhesive.

25. A method of polymerizing a polymerizable dental material, comprising initiating polymerization using a redox initiator system comprising:
a) at least three oxidizing agents comprising a peroxo group chosen from the group consisting of organic peroxides, organic hydroperoxides and persulfates,
b) at least one sulfinic acid and/or one sulfinic acid salt,
c) at least one photoinitiator, and
d) at least two tertiary amines, at least one of which exhibits a methyl benzoate group or ethyl benzoate group and at least one of which does not exhibit a methyl benzoate group or an ethyl benzoate group.

26. The method of claim 25, wherein the polymerizable dental material is a self-etching dental adhesive.
